(19) United States
(12) Patent Application Publication
(10) Pub. No.: US 2010/0331540 A1
(43) Pub. Date: Dec. 30, 2010

(54) METHOD FOR PRODUCING COMPOUND HAVING DEUTERATED AROMATIC RING OR HETEROCYCLIC RING

(76) Inventors: Haruki Shimodaira, Tsukuba-shi (JP); Toshifumi Abe, Tsukuba-shi (JP); Akira Miyazawa, Tsukuba-shi (JP); Yuji Kawanishi, Tsukuba-shi (JP); Taichi Abe, Tsukuba-shi (JP)

Correspondence Address: NIXON & VANDERHYE, PC, 901 NORTH GLEBE ROAD, 11TH FLOOR, ARLINGTON, VA, 22203, US

(21) Appl. No.: 12/865,240

(22) PCT Filed: Jan. 30, 2009
(86) PCT No.: PCT/JP2009/051643

§ 371 Date: Jul. 29, 2010

(30) Foreign Application Priority Data
Feb. 1, 2008 (JP) 2008-023130

(51) Int. Cl. C07D 241/46; C07D 279/18; C07D 231/12; C07D 277/20; C07D 233/56; C07D 215/04; C07D 213/06; C07D 209/04; C07C 209/68

(52) U.S. Cl. 544/35; 544/347; 546/173; 546/348; 548/202; 548/304.4; 548/335.1; 548/377.1; 548/490; 564/305; 564/433

(57) ABSTRACT

A method for producing a compound having a deuterated aromatic ring or heterocyclic ring according to the invention includes heating a compound having an aromatic ring or heterocyclic ring in the presence of heavy water, a transition metal and a metal which generates deuterium. As the metal which generates deuterium, at least one metal selected from the group consisting of aluminum, magnesium, zinc, iron, lead and tin is preferred. As the transition metal, at least one metal selected from the group consisting of platinum, palladium, ruthenium and rhodium is preferred. The heating is preferably carried out by microwave irradiation.

### TECHNICAL FIELD

The present invention relates to a method for producing a compound having a deuterated aromatic ring or heterocyclic ring. More specifically, the present invention relates to a method for producing a compound having a deuterated aromatic ring or heterocyclic ring by causing a deuterium atom to bond with an aromatic ring or heterocyclic ring in the compound.

Priority is claimed on Japanese Patent Application No. 2008-023130, filed Feb. 1, 2008, the content of which is incorporated herein by reference.

### BACKGROUND ART

A deuterium atom is one of the stable isotopes of hydrogen and has different physical properties from those of a protium atom. Accordingly, a deuterated compound not only exhibits different physical properties from those of the usual compound, but may also exhibit different chemical reactivity. Due to such characteristics, by deuterating a compound, it may be possible to provide the compound with functions which have not been available, and thus the development of various functional materials such as electronic materials and organic electroluminescence (EL) materials has been expected.

In addition, deuterated compounds have conventionally been used as an internal standard material in the microanalysis, such as mass spectrometry, of chemical substances. Accordingly, if a variety of deuterated compounds can be obtained, it is expected that great technical development can be achieved in the field of analytical science. For example, since pharmacokinetic analysis can be conducted by administration of a deuterated compound to a living body, improvements in the drug discovery technology and medical technology have been expected. In addition, as a familiar issue, the detection of residual agricultural chemicals in the food product is also a problem. In Japan, Regulation on Maximum Residue Limit (so-called positive list system) has been enforced since May 29, 2006, and the issue of food safety control will become more and more important in the future. Therefore, also for determination of the amount of residual agricultural chemicals, utilization of various deuterated compounds as internal standard materials has been expected.

From such circumstances, the development of a technology which enables the production of a desired deuterated compound simply and at low cost has been anticipated.

Many of those chemical substances to be analyzed in the field of medicine and agricultural chemicals have aromatic rings or heterocyclic rings which is a characteristic feature of those involved in the chemical reactions in vivo. Accordingly, the establishment of a technology for deuterating compounds having an aromatic ring or heterocyclic ring is of particular importance.

As a conventional method for deuterating compounds having an aromatic ring, for example, a method has been disclosed, in which an aromatic compound is deuterated under heating conditions using a palladium catalyst which has been activated in advance by hydrogen gas (refer to Non-Patent Document 1).

In addition, as a method for deuterating compounds having a heterocyclic ring, for example, a method has been disclosed, in which a compound having a heterocyclic ring is heated under reflux in a sealed state in a deuterated solvent in the presence of an activated metal catalyst (refer to Patent Document 1).

Further, a hydrogen-deuterium exchange reaction of triethylamine, which is an aliphatic compound, using an aluminum powder, a platinum carbon catalyst, heavy water and microwaves has been disclosed (refer to Non-Patent Document 2).

[Non-Patent Document 1] Christopher Hardacre, John D. Holbrey & S. E. Jane McMath, Chem. Commun., 2001, pp. 367-368.

[Non-Patent Document 2] “Microwave-assisted Chemical Process Technology”,

CMC Publishing, pp. 154-155.

[Patent Document I] PCT International Publication No. WO2004-046066 pamphlet

### DISCLOSURE OF INVENTION

### Problems to be Solved by the Invention

However, it is necessary to subject a palladium catalyst which has been activated by hydrogen gas to repeated freeze degassing before supplying to the deuteration reaction in the method described in Non-Patent Document 1, which makes the operation complicated.

In addition, it is necessary to bring a metal catalyst into contact with hydrogen gas or deuterium gas for activation before starting the reaction in the method described in Patent Document 1, making the operation complicated.

Furthermore, although the deuterium exchange of an aliphatic compound has been disclosed in Non-Patent Document 2, there is no disclosure of deuterium exchange in the compounds having an aromatic ring or heterocyclic ring, with completely different skeletons and reactivity levels.

The present invention takes the above circumstances into consideration, with an object of providing a method for producing a compound having a deuterated aromatic ring or heterocyclic ring, which can be applied to various raw material compounds and which can obtain a target product at high yield with simple and easy operations.

### Means for Solving the Problems

In order to solve the above-mentioned problems,

the present invention provides a method for producing a compound having a deuterated aromatic ring or heterocyclic ring, in which the compound having an aromatic ring or heterocyclic ring is heated in the presence of heavy water, a transition metal and a metal which generates deuterium.

In the present invention, it is preferable that the metal which generates deuterium be at least one metal selected from the group consisting of aluminum, magnesium, zinc, iron, lead and tin.

In addition, it is preferable that the transition metal be at least one metal selected from the group consisting of platinum, palladium, ruthenium and rhodium.

Further, the heating of the compound is preferably carried out by microwave irradiation.

Moreover, it is preferable to set the pressure of gas phase during the heating at 0.5 to 5 MPa.

Furthermore, it is preferable to make the ratio in terms of the amount of deuterium based on the total amount of deuterium and hydrogen in the reaction system equal to or more than the intended deuteration ratio of the compound.

### EFFECTS OF THE INVENTION

According to the present invention, compounds having a deuterated aromatic ring or heterocyclic ring can be produced at high yield with simple and easy operations by using various raw material compounds.

### BRIEF DESCRIPTION OF THE DRAWINGS

FIG. 1 is a diagram showing the ¹H-NMR spectra of phenazine-d₈ obtained in Example 1 (above) and of phenazine (below).

FIG. 2 is a diagram showing the GC-MS spectra of phenazine-dg obtained in Example 1 (above) and of phenazine (below).

FIG. 3 is a diagram showing the GC-MS spectra of 2,6-dimethylaniline-d₉ obtained in Example 8 (above) and of 2,6-dimethylaniline (below).

### BEST MODE FOR CARRYING OUT THE INVENTION

The present invention will be described in detail below.

In the present invention, by heating a compound having an aromatic ring or heterocyclic ring (hereafter, sometimes referred to as a “raw material compound”) in the presence of heavy water (D₂O), a transition metal and a metal which generates deuterium, a deuterated compound in which the aforementioned aromatic ring or heterocyclic ring is deuterated can be obtained.

As a raw material compound, a compound having either one or both of an aromatic ring and a heterocyclic ring can be used.

Deuteration of the aromatic ring or heterocyclic ring refers to a bonding of a deuterium atom to these rings. The term “deuterium atom” used herein refers to deuterium (D, ²H) or tritium (T, ³H), and the term “deuteration” refers to deuteration or tritiation. Further, the term “bonding” refers to a chemical bonding such as a covalent bonding. More specifically, deuteration of the aromatic ring or heterocyclic ring refers to, for example, substitution of the hydrogen atom bonded to the carbon atom or hetero atom constituting the ring structure of the aromatic ring or heterocyclic ring with a deuterium atom, or when there is a group bonded to the ring structure, substitution of the hydrogen atom constituting this group with a deuterium atom.

The number of deuterium atoms to be bonded can be adjusted by the amount of heavy water used, and appropriate adjustments may be made in accordance with the types of raw material compounds or the intended deuteration ratio of target products. Here, the term “deuteration ratio” refers to the proportion (%) of the number of hydrogen atoms in a deuterated compound which have been substituted with deuterium atoms based on the number of hydrogen atoms in a raw material compound which may be substituted with deuterium atoms. In those cases where the raw material compound only includes either one of the aromatic ring and heterocyclic ring, the hydrogen atoms which may be substituted with deuterium atoms refer to the hydrogen atoms bonded to the carbon atom or hetero atom constituting the ring structure of the aromatic ring or heterocyclic ring and the hydrogen atoms constituting a group that is bonded to the ring structure. In those cases where the raw material compound includes both the aromatic ring and heterocyclic ring, the hydrogen atoms which may be substituted with deuterium atoms refer to the hydrogen atoms bonded to the carbon atom or hetero atom constituting the ring structures of the aromatic ring and heterocyclic ring and the hydrogen atoms constituting a group that is bonded to these ring structures.

### (Heavy Water)

In the present invention, the amount of heavy water used may be appropriately adjusted in view of the types of raw material compounds, the intended deuteration ratio of target products, and the like. In addition, it is preferable to determine the amount of heavy water used so that the ratio of the amount of deuterium based on the total amount of deuterium and hydrogen in the reaction system becomes equal to or more than the intended deuteration ratio of the target product.

For example, when the intended deuteration ratio of the target product is 90%, the aforementioned ratio is preferably 91 to 95%. In addition, the aforementioned ratio is preferably adjusted appropriately in accordance with the intended deuteration ratio.

Note that the term “reaction system” used in the present invention refers to the reaction solution and gas phase portion in a reaction vessel.

For example, the deuteration ratio of the target product can be expressed by the formula:

B/A×100(%),

where A denotes the number of hydrogen atoms in a raw material compound which may be substituted with deuterium atoms and B denotes the number of hydrogen atoms in the deuterated target product which have been substituted with deuterium atoms.

Meanwhile, on the assumption that the deuteration reaction is carried out in heavy water, if the ratio of the volume of heavy water based on the volume inside the reaction vessel is within an ordinary range, most of the hydrogen atoms in the reaction system are originated from the raw material compound and the water (H₂O) mixed within the heavy water. Here, the phrase “within an ordinary range” refers to the cases excluding those where the volume of heavy water is considerably smaller than the volume inside the reaction vessel, and more specifically, for example, refers to the cases where above ratio is 5% or more.

Meanwhile, most of the deuterium atoms in the reaction system are originated from the heavy water. Although there is a possibility that deuterium atoms are present in the raw material compound as well as in the hydrogen gas in the air and water, the amount thereof is extremely low and is therefore negligible.

Accordingly, the amount I (mol) of hydrogen atoms in the reaction system can be approximated by the formula:

I=(X×A)+{Y×2×(100−Z)/100},

where X (mol) denotes the amount of raw material compound used, Y (mol) denotes the amount of heavy water used, and Z denotes the degree of deuterium enrichment in heavy water (i.e., the proportion of deuterium based on the total amount of the deuterium atoms and hydrogen atoms in heavy water, expressed in terms of atom %).

Meanwhile, the amount II (mol) of deuterium atoms in the reaction system can be approximated by the formula:

II=Y×2×Z/100.

Accordingly, the ratio III (%) in terms of the amount of deuterium based on the total amount of deuterium and hydrogen in the reaction system can be expressed by the formula:

III=II/(I+II)×100.

For example, when 0.01 mol of phenazine serving as a raw material compound and 2.75 mol of heavy water having a purity of 99.9 atom % are used, since A=8, X=0.01, Y=2.75 and Z=99.9 in this case, the following results can be obtained:

I=0.01×8+{2.75×2×(100−99.9)/100}=0.0855,

II=2.75×2×99.9/100=5.4945, and

III=5.4945/(0.0855+5.4945)×100=98.47(%).

In the present invention, it is preferable to adjust at least one of the aforementioned X, Y and Z so that the above ratio III of the deuterium amount becomes greater than the intended deuteration ratio of the target product (i.e., “B/A×100”).

As mentioned above, it should be noted that the example described here is excluding the cases where the volume of heavy water is considerably smaller than the volume inside the reaction vessel. On the other hand, even when the volume of heavy water is considerably small, the ratio of the amount of deuterium based on the total amount of deuterium and hydrogen in the reaction system can be adjusted without any problems, for example, by substituting the gas phase portion in the reaction vessel with an inert gas as described later, or by increasing the amount of heavy water used.

Suitable heavy water has a purity of, preferably at least 90 atom %, more preferably at least 95 atom %, and particularly preferably at least 99 atom %.

In addition, it is preferable to carry out the deuteration reaction by using heavy water as a solvent. When a solvent other than the heavy water is used in combination, it is preferable to use a solvent that does not contain a hydrogen atom.

Although it is not necessarily required to dissolve the raw material compound in heavy water, it is preferable to dissolve the raw material compound in heavy water by adjusting the reaction conditions or the like in order to carry out the deuteration reaction smoothly.

### (Transition Metal)

In the present invention, the term “transition metal” refers to the metals belonging to group III to group XI, and examples thereof include those that are known to have a catalytic function in the hydrogenation reaction. Among them, platinum, palladium, ruthenium and rhodium are preferred. More specifically, a transition metal supported on the surface of the activated carbon can be mentioned. As a catalyst containing such a transition metal, platinum-activated carbon (platinum-carbon), palladium-activated carbon (palladium-carbon), ruthenium-activated carbon (ruthenium-carbon) and rhodium-activated carbon (rhodium-carbon) are preferred, and platinum-activated carbon and palladium-activated carbon are particularly desirable.

The amount of transition metal used may be in a catalyst amount and can be appropriately adjusted. However, it is preferable that the amount be 0.05 to 10% by mass, more preferably 0.1 to 7% by mass, and particularly preferably 0.15 to 5% by mass, with respect to the compound having an aromatic ring or heterocyclic ring which serves as a raw material.

One type of transition metal may be used alone or two or more types of transition metals may be used in combination. In those cases where two or more types of transition metals are used in combination, the combination and ratio of these transition metals may be appropriately selected depending on the purpose.

In the present invention, as described later, deuterium is produced from heavy water due to the metal which generates deuterium, and the aforementioned transition metal is activated by the generated deuterium. Therefore, there is no need to activate the transition metal before the reaction starts.

(Metal which Generates Deuterium)

As a metal which generates deuterium, a metal that causes the deuterium generation upon contact with heavy water can be mentioned, and, for example, the metals known to cause the hydrogen generation upon contact with water can be used. Of these, preferred examples include aluminum, magnesium, zinc, iron, lead and tin, and aluminum, magnesium and zinc are more preferable, and aluminum is particularly desirable.

It is preferable to use a metal which generates deuterium in a powder form, since this form enables the increase of the contact surface with heavy water, when compared with other forms on the same mass basis.

The amount used of the metal which generates deuterium may be in a catalyst amount and can be appropriately adjusted. However, it is preferable that the amount be 1 to 80% by mass, more preferably 2 to 60% by mass, and particularly preferably 3 to 50% by mass, with respect to the raw material compound.

One type of the metal which generates deuterium may be used alone or two or more types of these metals may be used in combination. In those cases where two or more types of metals causing the deuterium generation are used in combination, the combination and ratio of these metals may be appropriately selected depending on the purpose.

### (Compound Having an Aromatic Ring or Heterocyclic Ring)

In the present invention, the compound having an aromatic ring or heterocyclic ring (i.e., the raw material compound) refers to a compound having at least one of an aromatic ring and a heterocyclic ring. Therefore, the compounds having both of an aromatic ring and a heterocyclic ring can also be used.

The aromatic ring may be either a monocyclic ring or a polycyclic ring, although a monocyclic ring is preferred. When the aromatic ring is a polycyclic ring, the ring is preferably bicyclic.

In the aromatic ring, although the number of carbon atoms constituting one ring structure is not particularly limited, it is preferably 5 to 7, more preferably 5 or 6, and most preferably 6.

Specific examples of the compound having an aromatic ring include benzene, toluene, o-xylene, m-xylene, p-xylene, phenol, o-cresol, m-cresol, p-cresol, pyrocatechol, resorcinol, hydroquinone, naphthalene, anthracene, phenanthrene, pyrene, perylene, naphthol, 2-naphthol, biphenyl, azulene, 1-anthrol, 2-anthrol, 9-anthrol, 1-phenanthrol, 2-phenanthrol, 3-phenanthrol, 4-phenanthrol, 9-phenanthrol, aniline, diphenylamine, 2,6-dimethylaniline, benzidine, benzoic acid, salicylic acid, 1-naphthoic acid, 2-naphthoic acid, phthalic acid, isophthalic acid, terephthalic acid, benzaldehyde, salicylic acid, 1-naphthaldehyde, 2-naphthaldehyde, phthalaldehyde, isophthalaldehyde and terephthalaldehyde.

Among these, preferred examples include benzene, toluene, diphenylamine and 2,6-dimethylaniline.

The heterocyclic ring has a hetero atom within the ring structure thereof. As the hetero atom, an oxygen atom, a nitrogen atom, a sulfur atom, a phosphorus atom or a silicon atom is preferable, and a nitrogen atom or a sulfur atom is more preferable. The heterocyclic ring may be one that has aromaticity or one that has no aromaticity, although those having aromaticity are preferred.

In the heterocyclic ring, although the number of hetero atoms in one ring structure depends on the total number of atoms constituting the ring structure and is not particularly limited, it is preferably 1 to 3 in general, and most preferably 1 or 2. In those cases where there is more than one hetero atom in one ring structure, all of these hetero atoms may be one type or some of them may be one type, or all of these hetero atoms may be different from one another. When there are several kinds of hetero atoms present in one ring structure, the combination is not particularly limited, although a combination of nitrogen atoms and sulfur atoms is preferred.

The heterocyclic ring may be either a monocyclic ring or a polycyclic ring, although a bicyclic ring or a tricyclic ring is preferred in the case of a polycyclic ring.

Specific examples of the compound having a heterocyclic ring include pyrrole, furan, thiophene, imidazole, 1-methylimidazole, 2-methylimidazole, 1,2-dimethylimidazole, 2-methyl-5-nitroimidazole, 1,2-dimethyl-5-nitroimidazole, 2-methyl-5-nitroimidazole-1-ethanol, pyrazole, oxazole, isoxazole, thiazole, isothiazole, 1,2,3-triazole, 1,2,4-triazole, pyridine, pyrazine, pyridazine, pyrimidine, 2H-pyran, 4H-pyran piperidine, piperazine, morpholine, quinoline, isoquinoline, purine, indole, benzimidazole, 2-hydroxybenzimidazole, 2-amino benzimidazole, benzothiophene, phenazine, phenothiazine, nicotinic acid, isonicotinic acid, nicotinaldehyde and isonicotinaldehyde.

Among these, preferred examples include indole, imidazole, 1-methylimidazole, 2-methylimidazole, 1,2-dimethylimidazole, 2-methyl-5-nitroimidazole, 1,2-dimethyl-5-nitroimidazole, 2-methyl-5-nitroimidazole-1-ethanol, 2-hydroxybenzimidazole, 2-aminobenzimidazole, pyridine, isoquinoline, pyrazole, benzimidazole, phenazine and phenothiazine.

As the compounds having an aromatic ring or the compounds having a heterocyclic ring, for example, those compounds specifically described above in which at least one hydrogen atom is substituted with a substituent may also be used. The number of hydrogen atoms to be substituted with a substituent is preferably 1 to 3, although it also depends on the types of aromatic rings or heterocyclic rings.

There are no particular limitations on the above substituent as long as the effects of the present invention are not impaired. Specific examples thereof include an alkyl group, an alkenyl group, an alkynyl group, an aryl group, an arylalkyl group, an alkoxy group, an aryloxy group, an alkoxyalkyl group, an aryloxyalkyl group, an alkoxycarbonylalkyl group, an alkoxycarbonyl group, an aryloxycarbonyl group, an alkylcarbonyloxyalkyl group, an alkylcarbonyloxy group, an arylcarbonyloxy group, a hydroxyalkyl group, a hydroxyaryl group, a hydroxyl group, a carboxyl group, an amino group, a cyano group, a nitro group and a halogen atom.

The alkyl group as the aforementioned substituent may be any of linear, branched or cyclic. The linear or branched alkyl group preferably has 1 to 5 carbon atoms, and specific examples thereof include a methyl group, an ethyl group, an n-propyl group, an isopropyl group, an n-butyl group, an isobutyl group, a sec-butyl group, a tert-butyl group and an n-pentyl group. Of such groups, those having 1 to 3 carbon atoms are more preferred, and a methyl group is particularly preferred. The cyclic alkyl group may be either a monocyclic group or a polycyclic group and preferably has 5 to 10 carbon atoms, more preferably 5 to 7 carbon atoms.

The alkenyl group and alkynyl group as the aforementioned substituent may be any of linear, branched or cyclic. The linear or branched alkenyl group and alkynyl group preferably have 2 to 4 carbon atoms. The cyclic alkenyl group or alkynyl group may be either a monocyclic group or a polycyclic group and preferably has 5 to 10 carbon atoms, more preferably 5 to 7 carbon atoms.

The aryl group as the aforementioned substituent may be either a monocyclic group or a polycyclic group, although a monocyclic group is preferred, and a phenyl group or a tolyl group is particularly desirable.

Examples of the arylalkyl group as the aforementioned substituent include the aforementioned alkyl group in which at least one hydrogen atom has been substituted with the aforementioned aryl group. The number of hydrogen atoms substituted with the aforementioned aryl group is preferably 1 or 2, and more preferably 1.

Examples of the alkoxy group as the aforementioned substituent include the aforementioned alkyl group having an oxygen atom bonded to the carbon atom therein.

Examples of the aryloxy group as the aforementioned substituent include the aforementioned aryl group having an oxygen atom bonded to the carbon atom therein.

Examples of the alkoxyalkyl group as the aforementioned substituent include the aforementioned alkyl group in which at least one hydrogen atom has been substituted with the aforementioned alkoxy group. The number of hydrogen atoms substituted with the aforementioned alkoxy group is preferably 1 or 2, and more preferably 1.

Examples of the aryloxyalkyl group as the aforementioned substituent include the aforementioned alkyl group in which at least one hydrogen atom has been substituted with the aforementioned aryloxy group. The number of hydrogen atoms substituted with the aforementioned aryloxy group is preferably 1 or 2, and more preferably 1.

Examples of the alkoxycarbonylalkyl group as the aforementioned substituent include the aforementioned alkoxyalkyl group in which a “—O—” moiety has been substituted with a “—O—C(═O)—” moiety (with the provision that the oxygen atom bonded to the carbon atom via a single bond is bonded to the alkyl group and the carbon atom is bonded to the alkylene group, respectively).

Examples of the alkoxycarbonyl group as the aforementioned substituent include the aforementioned alkoxy group having a carbonyl group bonded to the oxygen atom therein.

Examples of the aryloxycarbonyl group as the aforementioned substituent include the aforementioned aryloxy group having a carbonyl group bonded to the oxygen atom therein.

Examples of the alkylcarbonyloxyalkyl group as the aforementioned substituent include the aforementioned alkoxycarbonylalkyl group in which a “—O—C(═O)—” moiety has been substituted with a “—C(═O)—O—” moiety.

Examples of the alkylcarbonyloxy group as the aforementioned substituent include the aforementioned alkoxycarbonyl group in which a “—O—C(═O)—” moiety has been substituted with a “—C(═O)—O—” moiety.

Examples of the arylcarbonyloxy group as the aforementioned substituent include the aforementioned aryloxycarbonyl group in which a “—O—C(═O)—” moiety has been substituted with a “—C(═O)—O—” moiety.

Examples of the hydroxyalkyl group as the aforementioned substituent include the aforementioned alkyl group in which at least one hydrogen atom has been substituted with a hydroxyl group. The number of hydrogen atoms substituted with a hydroxyl group is preferably 1 or 2, and more preferably 1. Of such groups, those having 1 to 3 carbon atoms are preferred, and a hydroxyethyl group is particularly preferred.

Examples of the hydroxyaryl group as the aforementioned substituent include the aforementioned aryl group in which at least one hydrogen atom has been substituted with a hydroxyl group. The number of hydrogen atoms substituted with a hydroxyl group is preferably 1 or 2, and more preferably 1.

Examples of the halogen atom as the aforementioned substituent include a fluorine atom, a chlorine atom, a bromine atom and an iodine atom.

The compounds specifically described above having an aromatic ring or a heterocyclic ring and from which hydrogen atoms have been removed may be bonded with each other, via the atoms from which hydrogen atoms has been removed, thereby forming a structure, and a compound having such a structure may also be used, for example, as the raw material compound. In such cases, the combination of the compounds bonded with each other is not particularly limited, and the compounds may be selected from the group consisting of, for example, a compound having only an aromatic ring, a compound having only a heterocyclic ring, and a compound having an aromatic ring and a heterocyclic ring. In addition, although the number of the above compounds bonded with each other is not particularly limited, it is preferably 2 or 3, and most preferably 2.

Preferred examples of such raw material compounds include 2-(4-thiazoyl)benzimidazole, 1-phenylisoquinoline, 1-phenylpyrazole, p-tolylpyridine and phenylpyridine.

### (Other Reaction Conditions)

The deuteration reaction can be carried out by heating the compound having an aromatic ring or heterocyclic ring in the presence of heavy water, a transition metal and a metal which generates deuterium.

Any of the heating methods that are capable of setting the heating temperature within a desired range may be used, and specific examples thereof include heating using an oil bath, heating using an autoclave, and heating by microwave irradiation. Among these heating methods, heating by microwave irradiation is particularly preferred since it is highly effective in promoting the reaction.

The reason why the heating by microwave irradiation is highly effective in promoting the reaction is not yet clear, but is considered as follows. That is, the metal which generates deuterium not only generates deuterium due to the combined action with heavy water, but also forms an inert oxide film on the surface thereof. It is thought, however, that since at least a portion of the film is disrupted by the microwave action and the exposed metal surface regains the capacity to interact with heavy water, the efficiency for generating deuterium improves. This is also supported by the observation made in a similar experiment using water (H₂O) that the partial pressure of hydrogen (H₂) inside the reaction vessel increases, as compared to the case where an oil bath is used for heating, it is thought that because the amount of generated deuterium increases as described above, the transition metal is activated even more, and at least a portion of the passive state formed on the surface of the transition metal is also disrupted by the microwave action, thereby improving the catalytic capacity thereof. Furthermore, it is thought that since the reaction solution can be heated rapidly as well as uniformly by the microwave irradiation, the substitution of hydrogen atoms by deuterium atoms also proceeds rapidly.

Although the temperature during heating may be appropriately adjusted in view of the types and concentrations of the raw materials used, it is preferably within a range from 100 to 250° C., more preferably from 120 to 230° C., and particularly preferably 140 to 210° C.

Although the heating time may be appropriately adjusted in view of the types and concentrations of the raw materials used, the temperature during heating, the heating method, or the like, it is particularly desirable to adjust the heating time depending on the heating method.

For example, when the heating is conducted using an autoclave, the heating time is preferably from 10 to 50 hours, more preferably from 15 to 40 hours, and particularly preferably from 20 to 30 hours.

Further, when the heating is conducted by the microwave irradiation, the heating time is preferably from 0.3 to 18 hours, more preferably from 0.5 to 12 hours, and particularly preferably from 0.7 to 9 hours.

In those cases where other heating methods are employed such as the heating using an oil bath, it is preferable to set the heating time longer than that in the above case of heating using an autoclave.

In addition, it is preferable to apply pressure to the gas phase inside the reaction vessel during the heating. The pressure applied at this time is preferably from 0.5 to 5 MPa, more preferably from 0.7 to 3 MPa, and particularly preferably from 1 to 2 MPa. By making the pressure higher than the lower limit, a high level of reaction promoting effect can be achieved. On the other hand, by making the pressure lower than the upper limit, a high level of effect to suppress the decomposition of raw materials and target products can be achieved. In addition, if the pressure is lower than the above upper limit, a reaction apparatus exhibiting a high level of pressure resistance is no longer required, and thus the target products can be produced at low cost.

During the deuteration reaction, the gas phase portion inside the reaction vessel may be substituted with an inert gas. Here, examples of the inert gas include nitrogen gas, argon gas and helium gas. By substituting with an inert gas, the hydrogen and water in the air can be removed from the gas phase portion, and the ratio in terms of the amount of deuterium based on the total amount of deuterium and hydrogen in the reaction system can be further increased. Accordingly, even if, for example, the amount of heavy water used is suppressed to a low level, target products can be obtained at a high deuteration ratio.

Following the deuteration reaction, the obtained reaction solution may be directly used as it is depending on the intended purpose, or may be subjected to an appropriate post treatment if necessary so that the target product can be taken out for use. When a post treatment is carried out, the processes necessary for the treatment may be carried out by appropriately combining known methods such as extraction, concentration, filtration and pH adjustment. For example, the transition metal or the metal which generates deuterium can be simply removed by filtration. Known methods may also be employed when the target product is taken out. For example, crystals may be deposited using the reaction solution or the product thereof obtained due to the post treatment, followed by the filtration of the deposited crystals, or a target product may be separated by column chromatography.

The production method of the present invention can apply a batch system in which the deuteration reaction is carried out in the reaction vessel. In addition, a continuous system can also be applied, in which, for example, the deuteration reaction is conducted by filling in the reaction column with the transition metal and the metal which generates deuterium, and continuously supplying the compound having an aromatic ring or heterocyclic ring and heavy water to the heated reaction column through the pipe connected to the reaction column.

According to the present invention, compounds having a high deuteration ratio of aromatic ring or heterocyclic ring can be produced at high yield. Since the deuteration reaction can be conducted within a short period of time under mild conditions, decomposition of the raw material compounds and target products is inhibited, thereby suppressing the formation of byproducts. In addition, activation of the transition metal prior to the reaction is not required, heavy water rather than deuterium gas can be used as a deuterium source, the bubbling of gas or the like is also unnecessary, and the operation is also simple and easy. As described above, since low cost materials can be used and the operation is also simple and easy, target products can be produced at low cost. Furthermore, since various materials can be used as the raw material compounds, numerous types of deuterated compounds can be produced. Deuteration can be conducted not only on the aromatic ring or heterocyclic ring but also on the group bonded thereto.

### EXAMPLES

The present invention will be described below in further detail by means of specific working examples. However, the present invention is not limited in any way by the working examples shown below.

It should be noted that the laboratory equipment, analyzers and reagents used in the working examples shown below are as follows.

### (1) Laboratory Equipment

Microwave reaction apparatus:

The “Discover” microwave reactor (manufactured by CEM Corporation) in which the maximum capacity of the reaction vessel was 10 mL was used in an experiment where the scale thereof in terms of the amount of heavy water used was 3 mL.
Microwave reaction apparatus:

The Micro SYNTH Labstation (manufactured by Milestone Inc.) in which the maximum capacity of the reaction vessel was 80 mL was used in an experiment where the scale thereof in terms of the amount of heavy water used was 50 mL.
Organic synthesis reaction apparatus:

Organic Synthesizer ChemiStation PPV4060 manufactured by Tokyo Rikakikai Co., Ltd.

### (2) Analysis Equipment

GC-MS: SUN200 manufactured by JEOL Ltd.
NMR: NM-GSX270 manufactured by JEOL Ltd. DATUM Solution Business Operations

### (3) Reagents

Heavy water (deuterium oxide (99.9 atom % D)): manufactured by Isotec, Inc.
Palladium-activated carbon (5% Pd)): manufactured by Wako Pure Chemical Industries, Ltd.
Platinum-activated carbon (5% Pt)): manufactured by Wako Pure Chemical Industries, Ltd.
Aluminum powder (99.9%, 425 μm): Wako Pure Chemical Industries, Ltd.
Other reagents: manufactured by Tokyo Chemical Industry Co., Ltd.

Further, the identification of compounds and the calculation of deuteration ratio were conducted by the NMR and GC-MS measurements.

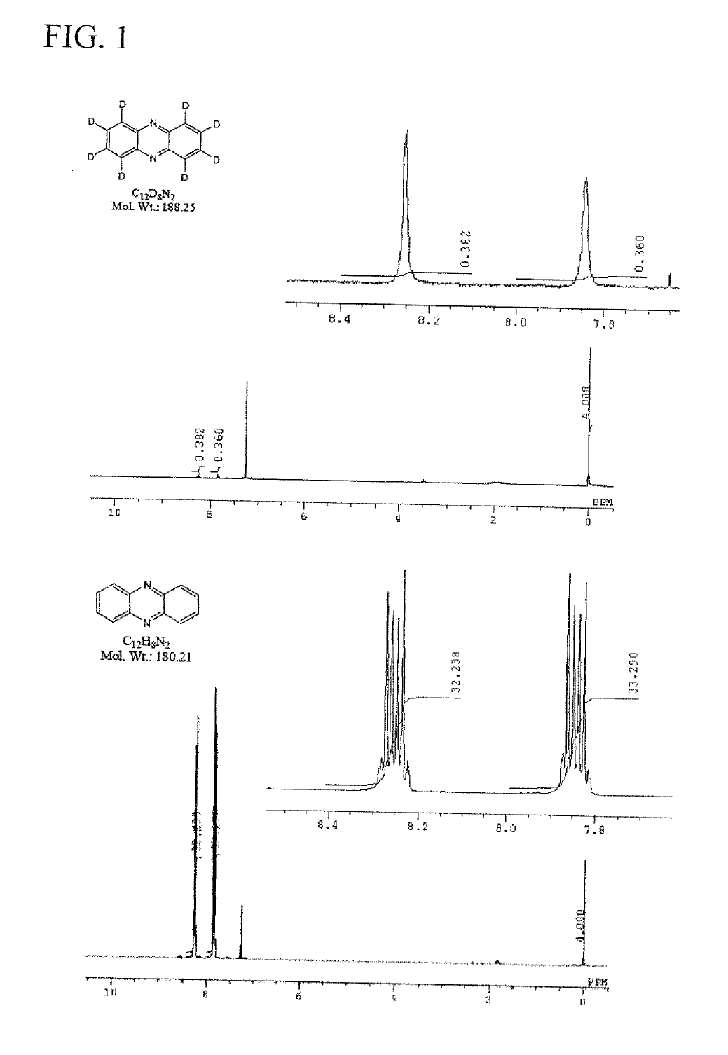

The identification of compounds by the NMR measurements was performed as follows. That is, a non-deuterated sample and a deuterated sample were subjected to ¹H-NMR measurements, and the successful deuteration was confirmed from the observation that the peak seen in the non-deuterated sample was either lost or considerably reduced in the deuterated sample. The data on NMR measurements made in Example 1 are shown in FIG. 1 as a specific example.

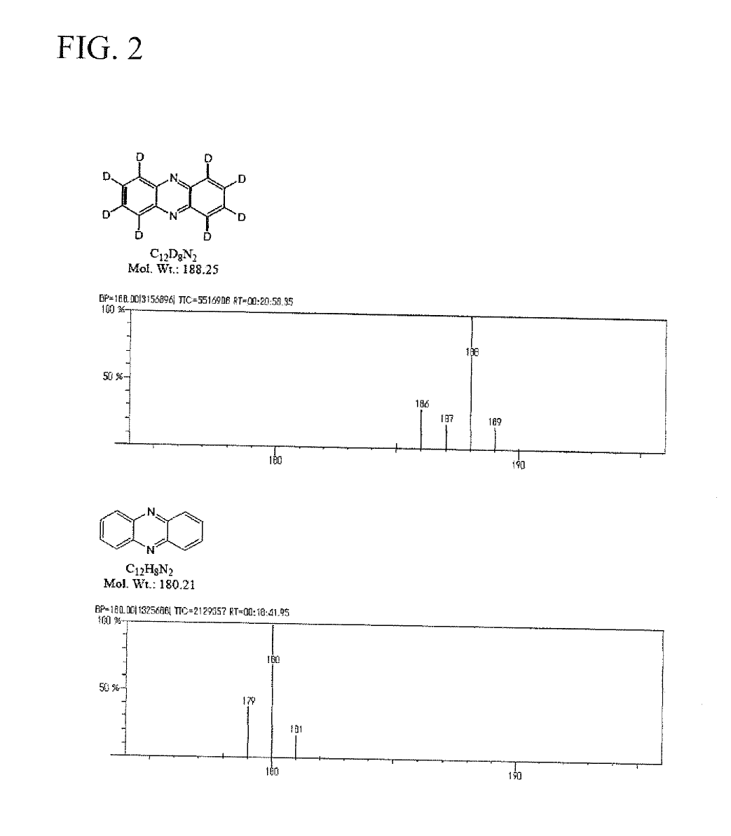

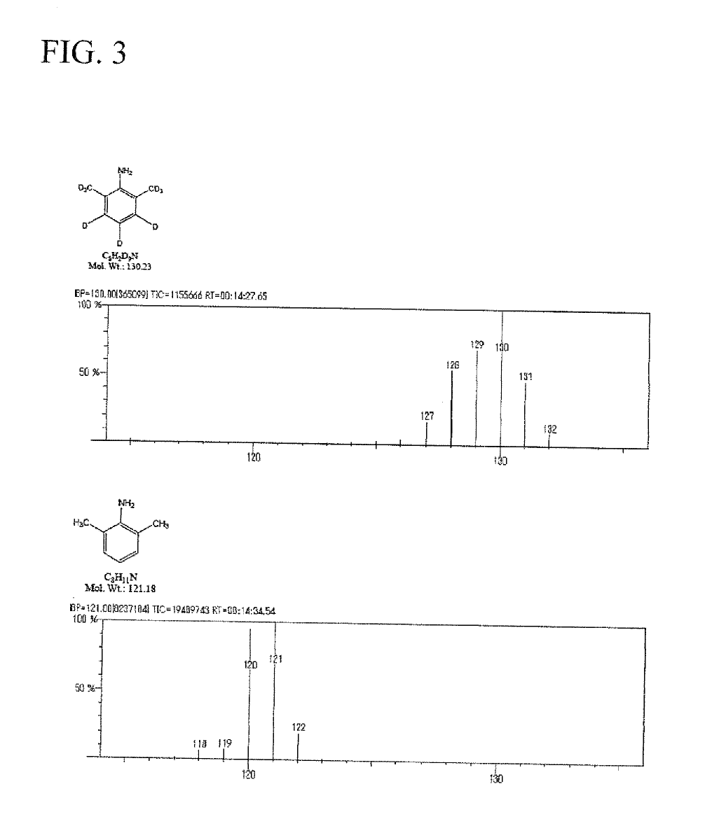

Further, the identification of compounds by the GC-MS measurements was conducted by subjecting a non-deuterated sample and a deuterated sample to GC-MS measurements, and confirming that the data supporting the change in molecular weight due to the deuteration were obtained. The data on GC-MS measurements made in Example 1 and GC-MS measurements made in Example 8 are shown in FIG. 2 and FIG. 3, respectively, as specific examples.

The methods for the NMR and GC-MS measurements and the method for calculating the deuteration ratio are shown below.

### (4) Calculation of Deuteration Ratio Based on the NMR Measurements

¹H-NMR measurements were conducted using an NMR solvent containing an internal standard material and dissolving a sample therein. The deuteration ratio was calculated on the basis of the integrated value of proton peaks of the internal standard material or intramolecular standard site.

### (5) Calculation of Deuteration Ratio Based on the GC-MS Measurements

GC-MS analyses were conducted on a non-deuterated sample and on a deuterated sample under the same conditions, and the deuteration ratio was calculated from the peak intensity ratio of the obtained fragment.

### Example 1

1.8 g of phenazine, 0.2 g of platinum-activated carbon (5%), and 0.2 g of aluminum powder were added to 50 ml of heavy water, and the resulting mixture was subjected to microwave irradiation at 200° C. for 180 minutes. The pressure applied during the reaction was 1.7 to 1.9 MPa. After being left to stand for cooling, the reaction mixture was extracted with dichloromethane, followed by the ¹H-NMR measurements (using deuterochloroform (hereafter, abbreviated as CDCl₃)) and GC-MS measurements (main peak (measured value); 188.00). As a result, the isolated yield of the deuterated compound was 87.8%, and the deuteration ratio was 98.9% (on average).

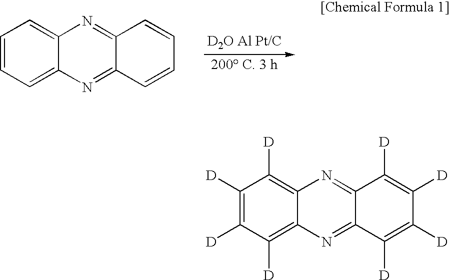

### Example 2

200 mg of phenothiazine, 50 mg of platinum-activated carbon (5%), and 50 mg of aluminum powder were added to 3 ml of heavy water, and the resulting mixture was subjected to microwave irradiation at 200° C. for 60 minutes. GC-MS measurements (main peak (measured value); 203.00) were carried out through the same operations as those described in Example 1. As a result, it was confirmed that the deuteration ratio of the deuterated compound was 106.0% (i.e., d4 form) (on average).

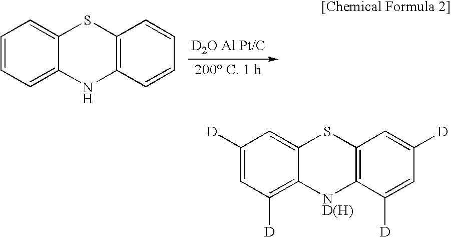

### Example 3

117 mg of indole, 50 mg of platinum-activated carbon (5%), and 50 mg of aluminum powder were added to 3 ml of heavy water, and the resulting mixture was subjected to microwave irradiation at 200° C. for 60 minutes. The pressure applied during the reaction was 1.4 to 1.7 MPa. GC-MS measurements (main peak (measured value); 123.00) were carried out through the same operations as those described in Example 1. As a result, it was confirmed that the deuteration ratio of the deuterated compound was 96.4% (on average).

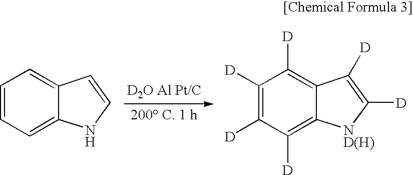

### Example 4

118 mg of benzimidazole, 50 mg of platinum-activated carbon (5%), and 50 mg of aluminum powder were added to 3 ml of heavy water, and the resulting mixture was subjected to microwave irradiation at 180° C. for 60 minutes. The pressure applied during the reaction was 1.4 to 1.7 MPa. ¹H-NMR measurements (using dimethyl sulfoxide-d₆ (hereafter, abbreviated as DMSO-d₆)) were carried out through the same operations as those described in Example 1. As a result, it was confirmed that the isolated yield of the deuterated compound was 96.9%. Moreover, it was confirmed that the deuteration ratios of the hydrogen atoms (1) to (3) were 91.6% (1), 78.9% (2), and 10.0% (3), respectively. Here, the hydrogen atoms (1) to (3) respectively correspond to the deuterium atoms (1) to (3) in the target product shown below.

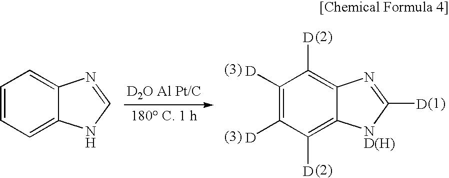

### Example 5

201 mg of 2-(4-thiazoyl)benzimidazole, 50 mg of platinum-activated carbon (5%), and 50 mg of aluminum powder were added to 3 ml of heavy water, and the resulting mixture was subjected to microwave irradiation at 200° C. for 60 minutes. The pressure applied during the reaction was 1.4 to 1.7 MPa. ¹H-NMR measurements (DMSO-d₆) were carried out through the same operations as those described in Example 1. As a result, it was confirmed that the isolated yield of the deuterated compound was 67.0%. Moreover, it was confirmed that the deuteration ratios of the hydrogen atoms (1) to (4) were 86.1% (1), 86.0% (2), 10.0% (3), and 0% (4), respectively. Here, the hydrogen atoms (1) to (4) respectively correspond to the deuterium atoms (1) to (4) in the target product shown below.

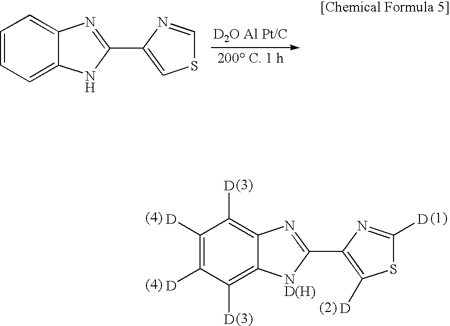

### Example 6

134 mg of 2-hydroxybenzimidazole, 50 mg of platinum-activated carbon (5%), and 50 mg of aluminum powder were added to 3 ml of heavy water, and the resulting mixture was subjected to microwave irradiation at 180° C. for 60 minutes. ¹H-NMR measurements (CDCl₃) on the obtained compound were carried out through the same operations as those described in Example 1. As a result, it was confirmed that the deuteration ratio of the deuterated compound was 99.0% (on average).

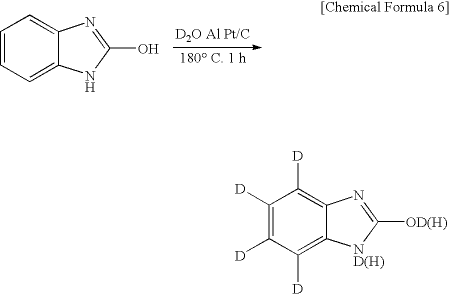

### Example 7

133 mg of 2-aminobenzimidazole, 50 mg of platinum-activated carbon (5%), and 50 mg of aluminum powder were added to 3 ml of heavy water, and the resulting mixture was subjected to microwave irradiation at 180° C. for 60 minutes. ¹H-NMR measurements (CDCl₃) on the obtained compound were carried out through the same operations as those described in Example 1. As a result, it was confirmed that the deuteration ratio of the deuterated compound was 99.0% (on average).

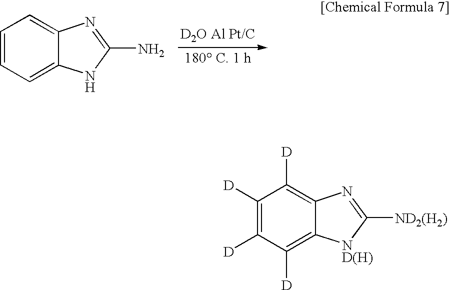

### Example 8

121 mg of 2,6-dimethylaniline, 50 mg of platinum-activated carbon (5%), and 50 mg of aluminum powder were added to 3 ml of heavy water, and the resulting mixture was subjected to microwave irradiation at 200° C. for 60 minutes. The pressure applied during the reaction was 1.5 to 1.7 MPa. ¹H-NMR measurements (DMSO-d₆) and GC-MS measurements (main peak (measured value); 130.00) on the obtained compound were carried out through the same operations as those described in Example 1. As a result, it was confirmed that the isolated yield of the deuterated compound was 96.9%. Moreover, it was confirmed that the deuteration ratios of the hydrogen atoms (1) and (2) were 97.9% ((1)-CD₃) and 80.5% ((2)-D), respectively. Here, the hydrogen atoms (1) and (2) respectively correspond to the deuterium atoms (1) and (2) in the target product shown below.

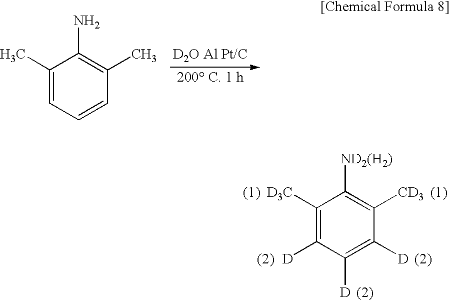

### Example 9

141 mg of 1,2-dimethyl-5-nitroimidazole, 10 mg of palladium-activated carbon (5%), and 10 mg of aluminum powder were added to 3 ml of heavy water, and the resulting mixture was subjected to microwave irradiation at 200° C. for 60 minutes. ¹H-NMR measurements (DMSO-d₆) on the obtained compound were carried out through the same operations as those described in Example 1. As a result, it was confirmed that the isolated yield of the deuterated compound was 57.2%. Moreover, it was confirmed that the deuteration ratios of the hydrogen atoms (1) and (2) were 56.7% ((1)-CD₃) and 99.0% ((2)-D), respectively. Here, the hydrogen atoms (1) and (2) respectively correspond to the deuterium atoms (1) and (2) in the target product shown below.

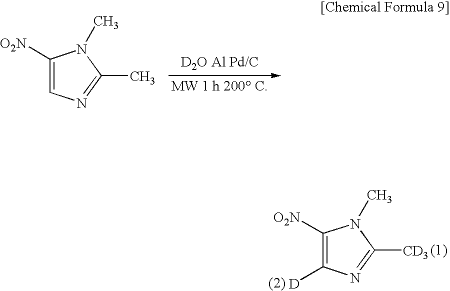

### Example 10

175 mg of 2-methyl-5-nitroimidazole-1-ethanol, 10 mg of palladium-activated carbon (5%), and 10 mg of aluminum powder were added to 3 ml of heavy water, and the resulting mixture was subjected to microwave irradiation at 200° C. for 60 minutes. ¹H-NMR measurements (DMSO-d₆) on the obtained compound were carried out through the same operations as those described in Example 1. As a result, it was confirmed that the isolated yield of the deuterated compound was 65.8%. Moreover, it was confirmed that the deuteration ratios of the hydrogen atoms (1) and (2) were 77.7% ((1)-CD₃) and 99.0% ((2)-D), respectively. Here, the hydrogen atoms (1) and (2) respectively correspond to the deuterium atoms (1) and (2) in the target product shown below.

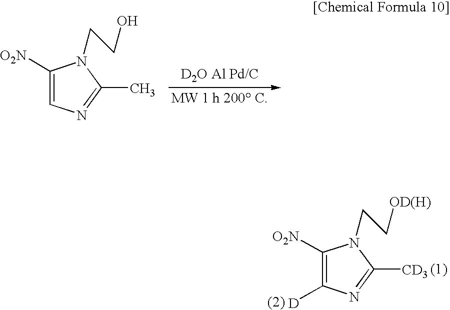

### Example 11

127 mg of 2-methyl-5-nitroimidazole, 10 mg of palladium-activated carbon (5%), and 10 mg of aluminum powder were added to 3 ml of heavy water, and the resulting mixture was subjected to microwave irradiation at 200° C. for 60 minutes. ¹H-NMR measurements (DMSO-d₆) on the obtained compound were carried out through the same operations as those described in Example 1. As a result, it was confirmed that the isolated yield of the deuterated compound was 83.7%. Moreover, it was confirmed that the deuteration ratios of the hydrogen atoms (1) and (2) were 6.0% ((1)-CD₃) and 99.0% ((2)-D), respectively. Here, the hydrogen atoms (1) and (2) respectively correspond to the deuterium atoms (1) and (2) in the target product shown below.

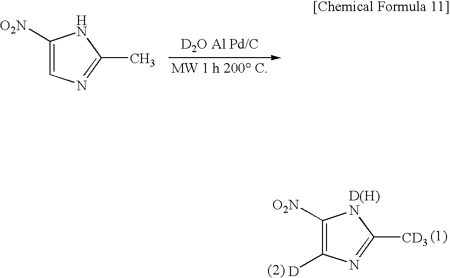

### Example 12

96 mg of 1,2-dimethylimidazole, 10 mg of palladium-activated carbon (5%), and 10 mg of aluminum powder were added to 3 ml of heavy water, and the resulting mixture was subjected to microwave irradiation at 200° C. for 60 minutes. ¹H-NMR measurements (DMSO-d₆) were carried out through the same operations as those described in Example 1. As a result, it was confirmed that the isolated yield of the deuterated compound was 87.5%. Moreover, it was confirmed that the deuteration ratios of the hydrogen atoms (1) to (3) were 79.3% ((1)-CD₃), 98.0% ((2)-D), and 98.0% ((3)-D), respectively. Here, the hydrogen atoms (1) to (3) respectively correspond to the deuterium atoms (1) to (3) in the target product shown below.

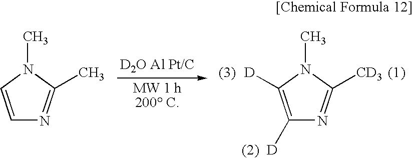

### Example 13

82 mg of 2-methylimidazole, 10 mg of palladium-activated carbon (5%), and 10 mg of aluminum powder were added to 3 ml of heavy water, and the resulting mixture was subjected to microwave irradiation at 200° C. for 60 minutes. ¹H-NMR measurements (CDCl₃) were carried out through the same operations as those described in Example 1. As a result, it was confirmed that the isolated yield of the deuterated compound was 93.4%. Moreover, it was confirmed that the deuteration ratios of the hydrogen atoms (1) to (3) were 96.5% ((1)-CD₃), 98.6% ((2)-D), and 98.6% ((3)-D), respectively. Here, the hydrogen atoms (1) to (3) respectively correspond to the deuterium atoms (1) to (3) in the target product shown below.

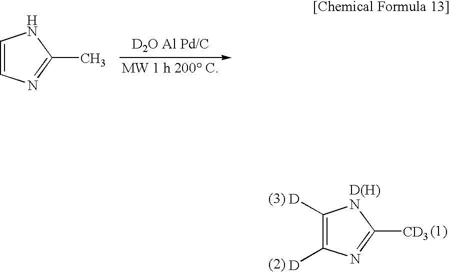

### Example 14

82 mg of 1-methylimidazole, 10 mg of palladium-activated carbon (5%), and 10 mg of aluminum powder were added to 3 ml of heavy water, and the resulting mixture was subjected to microwave irradiation at 200° C. for 60 minutes. ¹H-NMR measurements (CDCl₃) were carried out through the same operations as those described in Example 1. As a result, it was confirmed that the isolated yield of the deuterated compound was 88.0%. Moreover, it was confirmed that the deuteration ratios of the hydrogen atoms (1) to (3) were 94.0% ((1)-D), 98.0% ((2)-D), and 99.0% ((3)-D), respectively. Here, the hydrogen atoms (1) to (3) respectively correspond to the deuterium atoms (1) to (3) in the target product shown below.

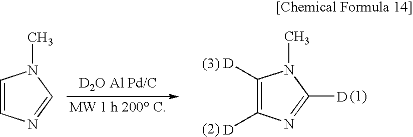

### Example 15

68 mg of imidazole, 10 mg of palladium-activated carbon (5%), and 10 mg of aluminum powder were added to 3 ml of heavy water, and the resulting mixture was subjected to microwave irradiation at 200° C. for 60 minutes. ¹H-NMR measurements (DMSO-d₆) were carried out through the same operations as those described in Example 1. As a result, it was confirmed that the isolated yield of the deuterated compound was 99.0%. Moreover, it was confirmed that the deuteration ratios of the hydrogen atoms (1) to (3) were 98.3% ((1)-D), 96.1% ((2)-D), and 96.1% ((3)-D), respectively. Here, the hydrogen atoms (1) to (3) respectively correspond to the deuterium atoms (1) to (3) in the target product shown below.

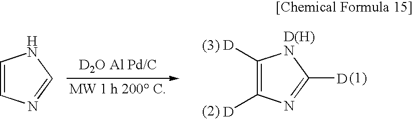

### Example 16

1 g of 1-phenylisoquinoline, 150 mg of platinum-activated carbon (5%), and 150 mg of aluminum powder were added to 25 ml of heavy water, and the resulting mixture was subjected to microwave irradiation at 180° C. for 300 minutes. After being left to stand for cooling, the reaction mixture was extracted with ether, followed by the ¹H-NMR measurements (CDCl₃). As a result, it was confirmed that the isolated yield of the deuterated compound was 90.0%, and the deuteration ratio was 80.0% (on average).

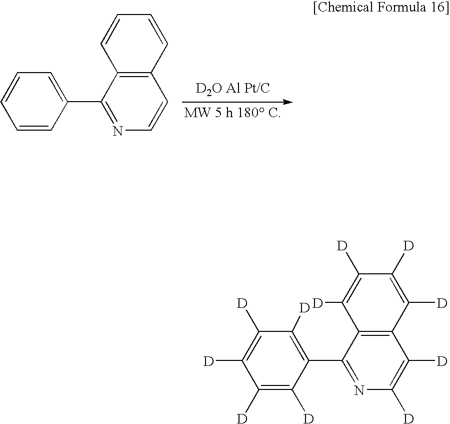

### Example 17

200 mg of 1-phenylpyrazole, 60 mg of platinum-activated carbon (5%), and 60 mg of aluminum powder were added to 3 ml of heavy water, and the resulting mixture was subjected to microwave irradiation at 150° C. for 60 minutes. ¹H-NMR measurements (CDCl₃) were carried out through the same operations as those described in Example 16. As a result, it was confirmed that the isolated yield of the deuterated compound was 95.0%, and the deuteration ratio was 80.0% (on average).

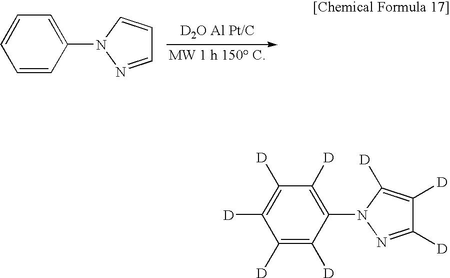

### Example 18

1 g of p-tolylpyridine, 150 mg of platinum-activated carbon (5%), and 150 mg of aluminum powder were added to 25 ml of heavy water, and the resulting mixture was subjected to microwave irradiation at 180° C. for 300 minutes. ¹H-NMR measurements (CDCl₃) were carried out through the same operations as those described in Example 16. As a result, it was confirmed that the isolated yield of the deuterated compound was 90.0%, and the deuteration ratio was 77.0% (on average).

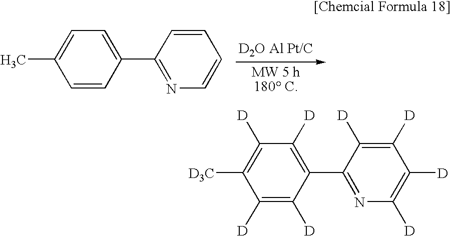

### Example 19

100 mg of diphenylamine, 50 mg of platinum-activated carbon (5%), and 4 mg of aluminum powder were added to 2 ml of heavy water, and the resulting mixture was subjected to microwave irradiation at 150° C. for 120 minutes. ¹H-NMR measurements (CD₂Cl₂) were carried out through the same operations as those described in Example 16. As a result, it was confirmed that the isolated yield of the deuterated compound was 93.0%, and the deuteration ratio was 95.0% (on average).

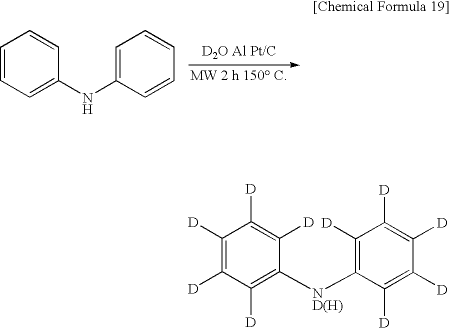

### Example 20

100 mg of phenylpyridine, 50 mg of platinum-activated carbon (5%), and 20 mg of aluminum powder were added to 2 ml of heavy water, and the resulting mixture was subjected to microwave irradiation at 150° C. for 60 minutes. GC-MS measurements (main peak (measured value); 164.00) were carried out through the same operations as those described in Example 16. As a result, it was confirmed that the isolated yield of the deuterated compound was 93.0%, and the deuteration ratio was 95.0% (on average).

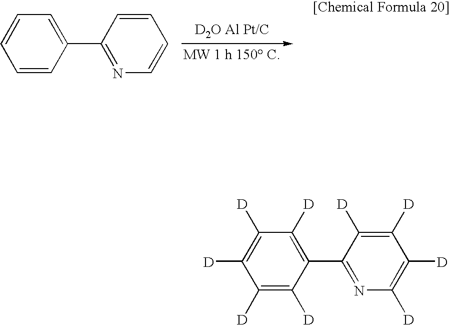

### Example 21

0.9 g of phenazine, 0.1 g of platinum-activated carbon (5%), and 0.1 g of aluminum powder were added to 50 ml of heavy water, and the resulting mixture was heated at 200° C. for 24 hours using an autoclave. The same operations and measurements as those described in Example 1 were conducted. As a result, it was confirmed that the isolated yield of the deuterated compound was 82.8%, and the deuteration ratio was 63.5%.

### Comparative Example 1

1.8 g of phenazine and 0.2 g of platinum-activated carbon (5%) were added to 50 ml of heavy water, and the resulting mixture was subjected to microwave irradiation at 200° C. for 60 minutes. The same operations and measurements as those described in Example 1 were conducted. As a result, it was confirmed that the isolated yield of the deuterated compound was 93.6%, and the deuteration ratio was 0%.

### INDUSTRIAL APPLICABILITY

The present invention can be applied to the microanalysis of chemical substances which requires an internal standard material, and is particularly suited for the pharmacokinetic analysis or the determination of residual agricultural chemicals. In addition, the present invention can also be applied to the electronic materials such as organic EL materials.

1. A method for producing a compound having a deuterated aromatic ring or heterocyclic ring, the method comprising: heating a compound having an aromatic ring or heterocyclic ring in the presence of heavy water, a transition metal and a metal which generates deuterium.

2. The method for producing a compound having a deuterated aromatic ring or heterocyclic ring according to claim 1, wherein the metal which generates deuterium is at least one metal selected from the group consisting of aluminum, magnesium, zinc, iron, lead and tin.

3. The method for producing a compound having a deuterated aromatic ring or heterocyclic ring according to claim 1, wherein the transition metal is at least one metal selected from the group consisting of platinum, palladium, ruthenium and rhodium.

4. The method for producing a compound having a deuterated aromatic ring or heterocyclic ring according to claim 1, wherein the heating is carried out by microwave irradiation.

5. The method for producing a compound having a deuterated aromatic ring or heterocyclic ring according to claim 1, wherein a pressure of gas phase during the heating is set at 0.5 to 5 MPa.

6. The method for producing a compound having a deuterated aromatic ring or heterocyclic ring according to claim 1, wherein a ratio in terms of an amount of deuterium based on the total amount of deuterium and hydrogen in a reaction system is equal to or more than an intended deuteration ratio of the compound.
